(19) United States
(12) Patent Application Publication
(10) Pub. No.: US 2021/0121541 A1
(43) Pub. Date: Apr. 29, 2021

(54) CGRP Antagonists and Clostridial Derivatives for the Treatment of Neuropsychiatric and Neurological Disorders

(71) Applicant: Allergan Pharmaceuticals International Limited, Dublin (IE)

(72) Inventors: Mitchell F. Brin, Newport Beach, CA (US); Andrew M. Blumenfeld, Del Mar, CA (US)

(21) Appl. No.: 16/921,502

(22) Filed: Jul. 6, 2020

Related U.S. Application Data
(60) Provisional application No. 62/870,916, filed on Jul. 5, 2019.

(51) Int. Cl. A61K 38/48; A61K 39/395; A61P 25/24; A61P 25/18; A61K 9/00

(52) U.S. Cl. CPC A61K 38/4893; A61K 39/3955; A61K 9/0019; A61P 25/18; A61P 25/24

(57) ABSTRACT

Methods for treating neurological or neuropsychiatric disorder and related symptoms by administering a CGRP antagonist and a Clostridial derivative are described.

### CROSS-REFERENCES TO RELATED APPLICATION

This application claims priority pursuant to 35 U. S.C. § 119(e) to U.S. Provisional Patent Application Ser. No. 62/870,916 filed Jul. 5, 2019, which is incorporated entirely herein by reference.

### FIELD

The application is related to medicaments and methods for treating neuropsychiatric and/or neurological disorders. The present application is directed to a medicament functioning as a calcitonin-gene-related-peptide (hereafter referred to as CGRP) antagonist administered by itself or in combination with a clostridial derivative, such as a botulinum toxin, for treating neuropsychiatric and/or neurological disorders and associated symptoms thereof.

### BACKGROUND

A neurological disorder is any disorder of the central and peripheral nervous system. Structural, biochemical or electrical abnormalities in the brain, the spine and the interconnecting nerves can result in a range of symptoms, such as paralysis, muscle weakness, poor coordination, loss of sensation, seizures, confusion, pain and altered levels of consciousness. The brain includes the dorsal end of the spinal cord, medulla, brain stem, pons, cerebellum, cerebrum and cortex. Examples of neurological disorders include migraine, epilepsy, chronic pain, post stroke pain, regional pain syndrome, phantom limb pain, and demyelinating disease pain.

A neuropsychiatric disorder is a neurological disturbance that is typically labeled according to four mental functions. For example, one group of neuropsychiatric disorders includes disorders of thinking and cognition, such as schizophrenia and delirium. A second group of neuropsychiatric disorders includes disorders of mood, such as affective disorders and anxiety. A third group of neuropsychiatric disorders includes disorders of social behavior, such as character defects and personality disorders. And a fourth group of neuropsychiatric disorders includes disorders of learning, memory, and intelligence, such as mental retardation and dementia. Thus, examples of neuropsychiatric disorders include schizophrenia, delirium, Alzheimer's disease, depression, mania, attention deficit disorders, drug addiction, dementia, agitation, apathy, anxiety, psychoses, personality disorders, bipolar disorders, obsessive-compulsive disorders, eating disorders, post-traumatic stress disorders, irritability, and di s-inhibition.

Current therapeutic treatments for neurological and neuropsychiatric disorders vary depending on the characteristics of the specific disorder, but can include pharmacological therapies or surgical procedures. Examples of surgical procedures include for example temporal lobectomy aimed to treat focal epilepsy or sympathectomy aimed to interrupt affected nerves of the sympathetic nervous system. Surgical procedures are invasive, carry inherent risks of complications, stroke or internal bleeding, and their effectiveness is uncertain. Pharmacological therapies, including anesthetics or pain-relief medications, anti-inflammatories, anti-depressants or narcotics often have several adverse side-effects. These side-effects may be attributed to the fact that the pharmaceutical agents are typically administered systemically, and therefore, the agents have a relatively non-specific action with respect to the various biological systems of the patient. Botulinum toxins have been administered to peripheral nerves such as the Trigeminal and Occipital nerves to treat conditions such as migraine. The administration has been associated with side effects such as neck pain, neck weakness and ptosis. These side effects are in part dose related. CGRP antagonists such as monoclonal antibodies to CGRP ligand or receptor have been administered subcutaneously or intravenously to treat conditions such as migraine. Neither of these treatments has a level of efficacy whereby every patient responds.

Thus, there is a need for an improved method of treating neurological and neuropsychiatric disorders.

### SUMMARY

The application provides methods for treating, alleviating or reducing the intensity or frequency of occurrence a neurological or neuropsychiatric disorder and/or symptoms thereof in a patient in need thereof by the use antagonists of calcitonin gene-related peptide (CGRP-antagonists), wherein the patient is concurrently undergoing treatment with a clostridial derivative. In some embodiments, the patient is administered CGRP-antagonist, and, a clostridial derivative.

In another aspect, the application provides methods for treating a neurological or neuropsychiatric disorder and/or symptoms thereof in patients by the use antagonists of calcitonin gene-related peptide (CGRP-antagonists), and optionally a clostridial derivative.

In some embodiments, the clostridial derivative is a recombinant clostridial toxin, a recombinant modified clostridial toxin, fragments of botulinum toxin, or targeted exocytosis modulators (TEMs), or combinations thereof. In some embodiments, the CGRP-antagonist is ubrogepant, atogepant, or a pharmaceutically acceptable salt, ester or prodrug thereof. In some embodiments, the CGRP-antagonist is an antibody is selected from galcanezumab, fremanezumab, eptinezumab, and erenumab.

In some embodiment, the CGRP antagonist, the clostridial derivative, or both are administered to a trigeminal, occipital, and/or cervical spinal nerve(s) of a patient. In some embodiments, the CGRP antagonist, the clostridial derivative, or both is administered to a trigeminal nerve. In some embodiments, the CGRP antagonist, the clostridial derivative, or both is administered to a cranial nerve, such as the vagal nerve. In some embodiments, the CGRP antagonist, the clostridial derivative, or both is administered to a peripheral nerve, such as the pudendal nerve, or a nerve root.

### DESCRIPTION

The application provides methods for treating, alleviating or reducing the intensity or frequency of occurrence of a neuropsychiatric or neurological disorder and/or symptoms thereof in a patient in need thereof by the use antagonists of calcitonin gene-related peptide (CGRP-antagonists), wherein the patient is concurrently undergoing treatment with a clostridial derivative. In some embodiments, the clostridial derivative is a botulinum toxin. In one embodiment, the clostridial derivative is onabotulinumtoxinA. In some embodiments, the methods relate to administering to the patient a CGRP-antagonist, and a botulinum toxin, preferably onabotulinumtoxinA.

In another aspect, the present disclosure provides a method of treating, alleviating or reducing the intensity or frequency of occurrence of a neuropsychiatric or neurological disorder and/or symptoms thereof in a patient in need thereof, the method comprises administering to the patient an antagonist of calcitonin gene-related peptide (CGRP-antagonists), and, optionally a clostridial derivative.

In some embodiments, the cGRP antagonist, and the clostridical derivative, or both is administered to a peripheral nerve, a cranial nerve, or combinations thereof, including but not limited to a trigeminal, occipital and/or cervical spinal nerve(s) of patient with a neurological disorder or neuropsychiatric disorder. In some embodiments, the neurological disorder is selected from the group consisting of migraine and other headache and facial pain disorders. In some embodiments, the neuropsychiatric disorder is selected from the group consisting of schizophrenia, depression, mania, or combinations thereof.

In some embodiments, the CGRP antagonist and the clostridial derivative are administered by similar administration routes. In one embodiment, the CGRP antagonist and the clostridial derivative are administered locally. In some embodiments, the CGRP antagonist and the clostridial derivative are administered by different administration routes. In some embodiments, the CGRP antagonist is administered systemically and the clostridial derivative is administered locally. In one embodiment, the CGRP antagonist is administered intravenously and the clostridial derivative is administered by local administration to a peripheral site as outlined in the section describing peripheral administration below. In some embodiments, the administration of the combination produces a synergistic effect relative to treatment with the CGRP antagonist or the clostridial derivative alone.

In some embodiments, the CGRP-antagonist is an anti-calcitonin gene-related peptide receptor antibody (anti-CGRP antibody) or antigen-binding fragment thereof. For example, the antibody can be selected from galcanezumab, fremanezumab, eptinezumab or erenumab. In some embodiments, the anti-CGRP antibody or fragment thereof is administered at a dosage that is about 20% or 30% or 40% or 50% or 60% or 70% or 80% lower than the recommended dosage for the anti-CGRP antibody monotherapy. In some embodiments, the anti-CGRP antibody or antigen-binding fragment thereof is administered to a peripheral nerve, a cranial nerve, or combinations thereof. CGRP-antagonists can be administered by any method that allows the antagonist to reach the intended targets.

For example, erenumab can be administered weekly, biweekly, monthly, every two months, every three months, every four months, every five months or every six months at a dosage of about 5 mg to about 500 mg.

Erenumab can be administered parenterally, subcutaneously or by peripheral administration. (Brauser D., Phase 3 STRIVE and ARISE Trials Show Efficacy, Safety for Erenumab in Migraine Prevention, Medscape Medical News, 2017) In one embodiment, erenumab is administered to trigeminal nerves.

In some embodiments, erenumab can be administered to the patient over the course of a set treatment period or indefinitely. (U.S. Patent Publication No. 20160311913) The treatment period can begin upon administration of a first dose of erenumab and continue while they have symptoms. The combination therapy with botulinum toxin includes administration of the clostridial derivative, such as a botulinum toxin, prior to, during or after the treatment period with erenumab. The treatment period can vary and in some embodiments, lasts only one day where the antibody is administered, and in some embodiments, can continue indefinitely while the patient continues to suffer from one or more symptoms of a neurological or neuropsychiatric disorder. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In some embodiment, erenumab is administered in a pharmaceutical composition comprising a buffer (preferably an acetate buffer), a surfactant (preferably polysorbate 20 or polysorbate 80), and a stabilizing agent (preferably sucrose). In one particular embodiment, the treatment period is at least about 6 months and produces a statistically significant reduction in the frequency, duration, or severity of at least one symptom of a neurological or neuropsychiatric disorder in the patient as compared to patients treated with erenumab or botulinum toxin alone.

In one embodiment, erenumab can be administered subcutaneously at a dose of about 5 mg to about 500 mg every one, two, three, four, five, six, seven, eight, nine or ten weeks.

In one embodiment, erenumab can be administered subcutaneously at a dose of about 10 mg to about 200 mg every one, two, three, four, five, six, seven, eight, nine or ten weeks.

In one embodiment, erenumab can be administered subcutaneously at a dose of about 25 mg to about 150 mg every one, two, three, four, five, six, seven, eight, nine or ten weeks.

In one embodiment, erenumab can be administered subcutaneously at a dose of about 90 mg to about 120 mg every one, two, three, four, five, six, seven, eight, nine or ten weeks.

In one embodiment, erenumab can be administered subcutaneously at a dose of about 50 mg to about 60 mg every one, two, three, four, five, six, seven, eight, nine or ten weeks.

In one embodiment, erenumab can be administered subcutaneously at a dose of about 70 mg every one, two, three, four, five, six, seven, eight, nine or ten weeks.

In one embodiment, erenumab can be administered subcutaneously at a dose of about 140 mg every one, two, three, four, five, six, seven, eight, nine or ten weeks.

In one embodiment, erenumab can be administered subcutaneously at a monthly dose of about 140 mg.

In one embodiment, erenumab can be administered subcutaneously at a monthly dose of about 70 mg.

In one embodiment, erenumab can be administered subcutaneously at a dose of about 140 mg every two months.

In one embodiment, erenumab can be administered subcutaneously at a dose of about 70 mg every two months.

In one embodiment, erenumab can be administered subcutaneously at a dose of about 140 mg every three months.

In one embodiment, erenumab can be administered subcutaneously at a dose of about 70 mg every three months.

In one embodiment, an anti-CGRP antibody galcanezumab can be administered weekly, biweekly, monthly, every two months, every three months, every four months, every five months or every six months at a dosage of about 5 mg to about 500 mg.

In some embodiments, galcanezumab can be administered to the patient over the course of a set treatment period. The treatment period can begin upon administration of a first dose galcanezumab and ends upon administration of a final dose of galcanezumab. The combination therapy with botulinum toxin includes administration of botulinum toxin prior to, during or after the treatment period with galcanezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of at least one symptom of a neurological or neuropsychiatric disorder in the patient as compared to patients treated with galcanezumab or botulinum toxin alone.

In one embodiment, galcanezumab is administered subcutaneously at a dose of about 10 mg to about 500 mg every one, two, three, four, five, six, seven, eight, nine or ten weeks.

In one embodiment, galcanezumab is administered subcutaneously at a dose of about 50 mg to about 300 mg every one, two, three, four, five, six, seven, eight, nine or ten weeks.

In one embodiment, galcanezumab is administered subcutaneously at a dose of about 75 mg to about 250 mg every one, two, three, four, five, six, seven, eight, nine or ten weeks.

In one embodiment, galcanezumab is administered subcutaneously at a dose of about 75 mg to about 100 mg every one, two, three, four, five, six, seven, eight, nine or ten weeks.

In one embodiment, galcanezumab is administered subcutaneously at a dose of about 150 mg to about 220 mg every one, two, three, four, five, six, seven, eight, nine or ten weeks.

In one embodiment, galcanezumab is administered subcutaneously at a dose of about 120 mg every one, two, three, four, five, six, seven, eight, nine or ten weeks.

In one embodiment, galcanezumab is administered subcutaneously at a dose of about 240 mg every one, two, three, four, five, six, seven, eight, nine or ten weeks.

In one embodiment, galcanezumab is administered subcutaneously at a monthly dose of about 240 mg.

In one embodiment, galcanezumab is administered subcutaneously at a monthly dose of about 120 mg.

In one embodiment, galcanezumab is administered subcutaneously at a dose of about 240 mg every two months.

In one embodiment, galcanezumab is administered subcutaneously at a dose of about 120 mg every two months.

In one embodiment, galcanezumab is administered subcutaneously at a dose of about 240 mg every three months.

In one embodiment, galcanezumab is administered subcutaneously at a dose of about 120 mg every three months.

In some embodiments, fremanezumab can be administered to the patient over the course of a set treatment period. (Silberstein, S. D., et. al., N Engl J Med 2017; 377:2113-22.) The treatment period can begin upon administration of a first dose fremanezumab and ends upon administration of a final dose of fremanezumab. The combination therapy with botulinum toxin includes administration of botulinum toxin prior to, during or after the treatment period with fremanezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of one symptom of a neurological or neuropsychiatric disorder in the patient as compared to patients treated with fremanezumab or botulinum toxin alone.

In one embodiment, fremanezumab is administered subcutaneously at a dose of about 100 mg to about 1000 mg every one, two, three, four, five, six, seven, eight, nine or ten weeks.

In one embodiment, fremanezumab is administered subcutaneously at a dose of about 150 mg to about 700 mg every one, two, three, four, five, six, seven, eight, nine or ten weeks.

In one embodiment, fremanezumab is administered subcutaneously at a dose of about 150 mg to about 500 mg every one, two, three, four, five, six, seven, eight, nine or ten weeks.

In one embodiment, fremanezumab is administered subcutaneously at a dose of about 150 mg to about 200 mg every one, two, three, four, five, six, seven, eight, nine or ten weeks.

In one embodiment, fremanezumab is administered subcutaneously at a dose of about 150 mg to about 500 mg every one, two, three, four, five, six, seven, eight, nine or ten weeks.

In one embodiment, fremanezumab is administered subcutaneously at a dose of about 225 mg every one, two, three, four, five, six, seven, eight, nine or ten weeks.

In one embodiment, fremanezumab is administered subcutaneously at a dose of about 450 mg every one, two, three, four, five, six, seven, eight, nine or ten weeks.

In one embodiment, fremanezumab is administered subcutaneously at a dose of about 675 mg every one, two, three, four, five, six, seven, eight, nine or ten weeks.

In one embodiment, fremanezumab is administered subcutaneously at a monthly dose of about 225 mg.

In one embodiment, fremanezumab is administered subcutaneously at a monthly dose of about 450 mg.

In one embodiment, fremanezumab is administered subcutaneously at a monthly dose of about 675 mg.

In one embodiment, fremanezumab is administered subcutaneously at a dose of about 225 mg every two months.

In one embodiment, fremanezumab is administered subcutaneously at a dose of about 450 mg every two months.

In one embodiment, fremanezumab is administered subcutaneously at a dose of about 225 mg every three months.

In one embodiment, fremanezumab is administered subcutaneously at a dose of about 450 mg every three months.

In one embodiment, fremanezumab is administered subcutaneously at a dose of about 675 mg every three months.

In some embodiments, eptinezumab can be administered to the patient over the course of a set treatment period. The treatment period can begin upon administration of a first dose eptinezumab and ends upon administration of a final dose of eptinezumab. The combination therapy with botulinum toxin includes administration of botulinum toxin prior to, during or after the treatment period with eptinezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of at least one symptom of a neurological or neuropsychiatric disorder in the patient as compared to patients treated with eptinezumab or botulinum toxin alone.

In one embodiment, eptinezumab is administered subcutaneously at a dose of about 50 mg to about 1000 mg every one, two, three, four, five, six, seven, eight, nine or ten weeks.

In one embodiment, eptinezumab is administered subcutaneously at a dose of about 100 mg to about 700 mg every one, two, three, four, five, six, seven, eight, nine or ten weeks.

In one embodiment, eptinezumab is administered subcutaneously at a dose of about 200 mg to about 500 mg every one, two, three, four, five, six, seven, eight, nine or ten weeks.

In one embodiment, eptinezumab is administered subcutaneously at a dose of about 250 mg to about 350 mg every one, two, three, four, five, six, seven, eight, nine or ten weeks.

In one embodiment, eptinezumab is administered subcutaneously at a dose of about 300 mg every one, two, three, four, five, six, seven, eight, nine or ten weeks.

In one embodiment, eptinezumab is administered subcutaneously at a monthly dose of about 100 mg.

In one embodiment, eptinezumab is administered subcutaneously at a monthly dose of about 200 mg.

In one embodiment, eptinezumab is administered subcutaneously at a monthly dose of about 300 mg.

In one embodiment, eptinezumab is administered subcutaneously at a dose of about 100 mg every two months.

In one embodiment, eptinezumab is administered subcutaneously at a dose of about 200 mg every two months.

In one embodiment, eptinezumab is administered subcutaneously at a dose of about 300 mg every two months.

In one embodiment, eptinezumab is administered subcutaneously at a dose of about 100 mg every three months.

In one embodiment, eptinezumab is administered subcutaneously at a dose of about 200 mg every three months.

In one embodiment, eptinezumab is administered subcutaneously at a dose of about 300 mg every three months.

In some embodiments, an antagonist of CGRP receptor can be administered in combination with a botulinum toxin. Preferably, the CGRP antagonist is selected from ubrogepant, atogepant, rimegepant or a pharmaceutically acceptable salt thereof.

In some embodiments, ubrogepant can be administered to the patient over the course of a set treatment period or indefinitely while the patient needs treatment. The treatment can be given over the life of the patent's disease continuously or intermittently as needed. In some embodiments, the treatment period includes a single administration of ubrogepant to a patient undergoing treatment with a botulinum toxin. In some embodiments, the treatment period continues where the patient is administered ubrogepant continuously or intermittently for a year or more. The treatment period can begin upon administration of a first dose of ubrogepant and continue until the patient is administered ubrogepant on a regular or intermittent basis. The combination therapy with botulinum toxin includes administration of botulinum toxin prior to, during or after the treatment period with ubrogepant. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of one symptom of a neurological or neuropsychiatric disorder in the patient as compared to patients treated with ubrogepant or botulinum toxin alone.

In some embodiments, ubrogepant is administered at an oral dose of about 5 to about 500 mg once, twice or three times a day.

In some embodiments, ubrogepant is administered at an oral dose of about 25 mg once, twice or three times a day.

In some embodiments, ubrogepant is administered at an oral dose of about 50 mg once, twice or three times a day.

In some embodiments, ubrogepant is administered at an oral dose of about 100 mg once, twice or three times a day.

In some embodiments, ubrogepant is administered at an oral dose of about 200 mg once, twice or three times a day.

In some embodiments, atogepant can be administered to the patient over the course of a set treatment period or indefinitely while the patient needs treatment. The treatment can be given over the life of the patent's disease continuously or intermittently as needed. In some embodiments, the treatment period includes a single administration of atogepant to a patient undergoing treatment with a botulinum toxin. In some embodiments, the treatment period continues where the patient is administered atogepant continuously or intermittently for a year or more. The treatment period can begin upon administration of a first dose atogepant and continue until the patient is administered atogepant on a regular or intermittent basis. The combination therapy with botulinum toxin includes administration of botulinum toxin prior to, during or after the treatment period with atogepant. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of at least one symptom of a neurological or neuropsychiatric disorder in the patient as compared to patients treated with atogepant or botulinum toxin alone.

In some embodiments, atogepant is administered at an oral dose of about 5 to about 500 mg once, twice or three times a day.

In some embodiments, atogepant is administered at an oral dose of about 25 mg once, twice or three times a day.

In some embodiments, atogepant is administered at an oral dose of about 50 mg once, twice or three times a day.

In some embodiments, atogepant is administered at an oral dose of about 100 mg once, twice or three times a day.

In some embodiments, atogepant is administered at an oral dose of about 200 mg once, twice or three times a day.

In some embodiments, rimegepant can be administered to the patient over the course of a set treatment period or indefinitely while the patient needs treatment. The treatment can be given over the life of the patent's disease continuously or intermittently as needed. In some embodiments, the treatment period includes a single administration of rimegepant to a patient undergoing treatment with a botulinum toxin. In some embodiments, the treatment period continues where the patient is administered rimegepant continuously or intermittently for a year or more. The treatment period can begin upon administration of a first dose rimegepant and continue until the patient is administered rimegepant on a regular or intermittent basis. The combination therapy with botulinum toxin includes administration of botulinum toxin prior to, during or after the treatment period with rimegepant. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of at least one symptom of a neurological or neuropsychiatric disorder in the patient as compared to patients treated with rimegepant or botulinum toxin alone.

In some embodiments, rimegepant is administered at an oral dose of about 5 to about 500 mg once, twice or three times a day.

In some embodiments, rimegepant is administered at an oral dose of about 25 mg once, twice or three times a day.

In some embodiments, rimegepant is administered at an oral dose of about 50 mg once, twice or three times a day.

In some embodiments, rimegepant is administered at an oral dose of about 100 mg once, twice or three times a day.

In some embodiments, rimegepant is administered at an oral dose of about 200 mg once, twice or three times a day.

In some embodiments, rimegepant is administered at an oral dose of about 5 to about 500 mg once, twice or three times a day.

In some embodiments, rimegepant is administered at an oral dose of about 25 mg once, twice or three times a day.

In some embodiments, rimegepant is administered at an oral dose of about 50 mg once, twice or three times a day.

In some embodiments, rimegepant is administered at an oral dose of about 100 mg once, twice or three times a day.

In some embodiments, rimegepant is administered at an oral dose of about 200 mg once, twice or three times a day.

In some embodiments, the combination therapy with a clostridial derivative, such as a botulinum toxin, reduces the frequency, severity and/or duration of at least one symptom of a neurological or neuropsychiatric disorder in patients in need thereof. In some embodiments, the combination therapy reduces the frequency of one or more symptoms or side effects of migraine; for example, sinusitis, nausea or nasopharangytis, in comparison with a patient undergoing treatment with CGRP-antagonist or botulinum toxin treatment alone. In some embodiments, the frequency of symptoms associated with one or more stages of migraine selected from prodrome, aura, headache or postdrome is reduced. For example, one or more frequency of symptoms associated with prodrome stage of migraine selected from photophobia, appetite changes, cognition and concentration difficulties, cold extremities, diarrhea or other bowel changes, excitement or irritability, fatigue, frequent urination, memory changes, weakness, yawning and stretching is reduced. In some embodiments, one or more frequency of symptoms associated with aura stage of migraine selected from seeing bright spots or flashes of light, vision loss, seeing dark spots, tingling sensations, speech problems, aphasia, and tinnitus is reduced. In some embodiments, one or more frequency of symptoms associated with attack stage of migraine selected from photophobia, gastric stasis, pulsating or throbbing pain on one or both sides of the head, extreme sensitivity to light, sounds, or smells, worsening pain during physical activity, nausea and vomiting, abdominal pain or heartburn, loss of appetite, lightheadedness, blurred vision, and fainting is reduced.

In some embodiments, the combination therapy with CGRP-antagonists and a clostridial derivative described herein is administered to a patient undergoing treatment with one or more additional medications for the treatment of a neurological or neuropsychiatric disorder and related symptoms. For example, the patient can be undergoing treatment with one or more additional medications selected from opioid analgesics, Cox-2 inhibitors, barbiturates, sodium channel modulators, potassium channel modulators, calcium channel blockers, local anesthetics, monoamine oxidase inhibitors, leukotriene LTD4 receptor blocker, substance P antagonists, 5-HT3 antagonists and NMDA antagonists. For example, the additional medication can be selected from aspirin, ibuprofen, naproxen, acetaminophen, diclofenac, flurbiprofen, meclofenamate, isometheptene, indomethacin; codeine, morphine, hydrocodone, acetyldihydrocodeine, oxycodone, oxymorphone, papaverine, fentanyl, alfentanil, sufentanil, remifentanyl, tramadol, prochlorperazine, celecoxib, rofecoxib, meloxicam, piroxicam, JTE-522, L-745,337, NS398, deracoxib, valdecoxib, iumiracoxib, etoricoxib, parecoxib, 4-(4-cyclohexyl-2-methyloxazol-5-yl)-2 fluorobenzenesulfonamide, (2-(3,5-difluorophenyl)-3-(4-(methylsulfonyl)phenyl)-2 cyclopenten-1-one, N-[2-(cyclohexyloxy)-4-nitrophenyl]methanesulfonamide, 2-(3,4 difluorophenyl)-4-(3-hydroxy-3-methylbutoxy)-5-[4-(methylsulfonyl) phenyl]-3(2H) pyridazinone, 2-[(2,4-dichloro-6-methylphenyl) amino]-5-ethyl-benzeneacetic acid, (3Z) 3-[(4-chlorophenyl) [4-(methyl sulfonyl)phenyl] methylene]dihydro-2(3H)-furanone, (S)-6,8-dichloro-2-(trifluoromethyl)-2H-1-benzopyran-3-carboxylic acid, amobarbital, butalbital, cyclobarbital, pentobarbital, allobarbital, methylphenobarbital, phenobarbital, secobarbital, vinylbital, verapamil, ciltiazem, Nifedipine, lidocaine, tetracaine, prilocaine, bupivicaine, mepivacaine, etidocaine, procaine, benzocaine, phehelzine, isocarboxazid, dichloralphenazone, nimopidine, metoclopramide, capsaicin receptor agonists, captopril, tiospirone, a steroid, caffeine, metoclopramide, domperidone, scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth sub salicylate, buclizine, cinnarizine, cyclizine, diphenidol, dolasetron, domperidone, dronabinol, droperidol, haloperidol, metoclopramide, nabilone, thiethylperazine, trimethobenzemide, and eziopitant, Meclizine, domperidone, ondansetron, tropisetron granisetron dolasetron, hydrodolasetron, palonosetron, alosetron, cilansetron, cisapride, renzapride metoclopramide, galanolactone, phencyclidine, ketamine, dextromethorphan, and isomers, pharmaceutically acceptable salts, esters, conjugates, or prodrugs thereof.

In some embodiments, the clostridial derivative is onabotulinumtoxinA and is administered at a dose of about 1 unit, about 2 units, about 3 units, about 4 units, about 5 units, about 6 units, about 7 units, about 8 units, about 9 units or about 10 units. The frequency of administration can be once every one, two, three, four, five, six, seven, eight, nine, ten, eleven, twelve, thirteen, fourteen, fifteen or sixteen weeks.

In some embodiments, onabotulinumtoxinA is administered at a dose of about 10 unit, about 15 units, about 20 units, about 25 units, about 30 units, about 40 units, about 45 units, about 50 units, about 55 units or about 60 units. The frequency of administration can be once every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about of about 25 unit, about 50 units, about 75 units, about 100 units, about 125 units, about 150 units, about 175 units, about 200 units, about 225 units or about 250 units every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about dose of about 1 to about 1,000 units. The frequency of administration can be once every one, two, three, four, five, six, seven, eight, nine, ten, eleven, twelve, thirteen, fourteen, fifteen, sixteen, seventeen, eighteen, nineteen or twenty weeks, or more. In some embodiments, the frequency of administration can be once every one, two, three, four, five, six, seven, eight, nine, ten, eleven, twelve, thirteen, fourteen, fifteen, sixteen, seventeen, eighteen months, or more. In some embodiments, the clostridial derivative is onabotulinumtoxinA and is administered at a dose of about 1 to about 100 units every one, two, three, four, five, six, seven, eight, nine, ten, eleven, twelve, thirteen, fourteen, fifteen or sixteen weeks.

In some embodiments, the clostridial derivative is onabotulinumtoxinA and is administered at a dose of about 10 to about 50 units. The frequency of administration can be once every one, two, three, four, five, six, seven, eight, nine, ten, eleven, twelve, thirteen, fourteen, fifteen or sixteen weeks.

The clostridial derivative can be administered by many different methods including peripheral, parenteral, subcutaneous, intravenous, intraperitoneal, intracerebral, intralesional, intramuscular, intraocular, intraarterial interstitial infusion and implanted delivery device. In some embodiments, the clostridial derivative is administered to a nerve. In some embodiments, the clostridial derivative is administered to a suture line. In one embodiment, the clostridial derivative is onabotulinumtoxinA.

In some embodiments, onabotulinumtoxinA is administered parenterally.

In some embodiments, onabotulinumtoxinA is administered topically.

In some embodiments, onabotulinumtoxinA is administered subcutaneously or intramuscularly.

In some embodiments, onabotulinumtoxinA is administered intradermally.

In some embodiments, onabotulinumtoxinA is administered subcutaneously once every month or two months.

In some embodiments, onabotulinumtoxinA is administered at a dose of about 155 units.

The effective amount of the clostridial derivative can be measured in mass units (e.g. in ng or mg). The effective dose in weight units can be determined based on the intended effect. For example, the effective weight can be determined based on the amount of clostridial derivative required to have a therapeutic effect on the muscle or a sensory effect. In some embodiments, the clostridial derivative can be administered at a dose of about 0.001 ng to about 1000 ng, preferably about 0.001 ng to about 500 ng, preferably about 0.01 ng to about 250 ng, preferably about 0.1 ng to about 150 ng, preferably about 1 ng to about 100 ng, preferably about 1 ng to about 10 ng. For example, onabotulinumtoxinA can be administered at a dose of about 1 ng, 2 ng, 3 ng, 4 ng, 5 ng, 6 ng, 7 ng, 8 ng, 9 ng or 10 ng.

In some embodiments, the CGRP-antagonist can be administered orally, sublingually, transdermally, subcutaneously, intravenously, intradermally or intramuscularly.

In one embodiment, the CGRP-antagonist can be administered intravenously. The intravenous formulation can contain a tonicity modifier to avoid crenation or hemolysis of red blood cells, and/or to mitigate or avoid pain and discomfort to the patient. Preferably, the formulation to be administered to the patient has an effective osmotic pressure that is approximately the same as that of the blood of the patient. Tonicity modifiers can be non-ionic tonicity modifiers such as glycerol, sorbitol, mannitol, sucrose, propylene glycol or dextrose, or a mixture thereof. Preferably the non-ionic tonicity modifier is dextrose, sucrose or mannitol, or a mixture thereof. Aqueous pharmaceutical formulations for intravenous administration generally can have a pH of from 3 to 9.

Stable liquid or solid pharmaceutical composition comprising a clostridial toxin derivative, a disaccharide, a surfactant and an antioxidant can be used in combination with CGRP-antagonists.

CGRP is a member of the calcitonin family of peptides, which in human exists in two form, α-CGRP and β-CGRP. α-CGRP and β-CGRP vary by three amino acids, have similar activities and exhibit differential distribution. At least two CGRP receptor subtypes may also account for differential activities. CGRP is produced in both peripheral and central neurons, and released by the trigeminal nerve. CGRP has been shown to be a potent vasodilator in the periphery, where CGRP-containing neurons are closely associated with blood vessels. CGRP-mediated vasodilatation is also associated with neurogenic inflammation, as part of a cascade of events that results in extravasation of plasma and vasodilation of the microvasculature and is present in migraine. CGRP is released by sensory nerves, e.g. the trigeminal nerve, which innervates part of the skin of the face. The trigeminal nerve has three major branches, a number of smaller branches and is the great sensory nerve of the head and neck, carrying touch, temperature, pain, and proprioception (position sense) signals from the face and scalp to the brainstem. Trigeminal sensory fibers originate in the skin, course toward the trigeminal ganglion (a sensory nerve cell body), pass through the trigeminal ganglion, and travel within the trigeminal nerve to the sensory nucleus of the trigeminal nerve located in the brainstem.

The three major branches of the trigeminal nerve are the ophthalmic (V₁, sensory), maxillary (V₂, sensory) and mandibular (V₃, motor and sensory) branches. The large trigeminal sensory root and smaller trigeminal motor root leave the brainstem at the midlateral surface of pons. The sensory root terminates in the largest of the cranial nerve nuclei which extends from the pons all the way down into the second cervical level of the spinal cord. The sensory root joins the trigeminal or semilunar ganglion between the layers of the dura mater in a depression on the floor of the middle crania fossa. The trigeminal motor root originates from cells located in the masticator motor nucleus of trigeminal nerve located in the midpons of the brainstem. The motor root passes through the trigeminal ganglion and combines with the corresponding sensory root to become the mandibular nerve. It is distributed to the muscles of mastication, the mylohyoid muscle and the anterior belly of the digastric. The three sensory branches of the trigeminal nerve emanate from the ganglia to form the three branches of the trigeminal nerve. The ophthalmic and maxillary branches travel in the wall of the cavernous sinus just prior to leaving the cranium. The ophthalmic branch travels through the superior orbital fissure and passes through the orbit to reach the skin of the forehead and top of the head. The maxillary nerve enters the cranium through the foramen rotundum via the pterygopalatine fossa. Its sensory branches reach the pterygopalatine fossa via the inferior orbital fissure (and supply sensation to the face, cheek and upper teeth) and pterygopalatine canal (and supply sensation to the soft and hard palate, nasal cavity and pharynx). There are also meningeal sensory branches that enter the trigeminal ganglion within the cranium. The sensory part of the mandibular nerve is composed of branches that carry general sensory information from the mucous membranes of the mouth and cheek, anterior two-thirds of the tongue, lower teeth, skin of the lower jaw, side of the head and scalp and meninges of the anterior and middle cranial fossae.

The sensory nuclei of the trigeminal nerve are located within the brainstem, in the dorsolateral pons. The mesencephalic tract and the motor nucleus of the trigeminal nerve lie more medially. The superior cerebellar peduncle lies posteriorly. It is continuous inferiorly with the spinal nucleus of the trigeminal nerve that extends into the medulla. Superiorly, the sensory nuclei on each side are continuous with the mesencephalic nucleus.

Importantly, the sensory nuclei of the trigeminal nerve receive afferent (sensory input) fibers from: (1) the trigeminal nerve ophthalmic division (e.g. general sensation from supraorbital area, cornea, iris, ethmoid sinuses), (2) trigeminal nerve maxillary division (e.g. sensation from temple, cheek, oral cavity, upper pharynx), and (3) trigeminal nerve mandibular division (e.g. sensation from middle cranial fossa, inner cheek, anterior two thirds of the tongue, chin), (4) facial nerve (e.g. general sensation from external auditory meatus), (5) glossopharyngeal nerve (e.g. general sensation from middle ear, tonsils, oropharynx, posterior one third of the tongue), (6) vagus nerve (auricular, meningeal, internal laryngeal and recurrent laryngeal branches).

Thus, primary neurons in the trigeminal ganglion synapse on the main sensory trigeminal nucleus and on the spinal trigeminal nucleus in the brainstem. The spinal nucleus of the trigeminal system extends to the upper cervical spine, where connections with cervical dermatomes exist. These dermatomes are innervated by the cervical sensory rami and the occipital nerves, which have sensory branches from C2 to C5. The trigeminal nerve also innervates stretch receptors in the muscles of mastication. The cell bodies of these neurons are in the mesencephalic trigeminal nucleus in the midbrain and pons).

As indicated by FIG. 1, the ascending (afferent) second order trigeminal neurons from the main sensory trigeminal nucleus, and the ascending second order neurons from the spinal trigeminal nucleus ascend and synapse in the thalamus. Projections from the thalamus are to the facial representation of the sensory cortex. Central projections from the mesencephalic trigeminal nucleus are to the motor cortex. Thalamic projections to the sensory cortex follow a somatopic organization. The hand and face have disproportionately greater representation on a homunculus map. This body map is not static, but dynamically controlled by the pattern of use, with increased use leading to increased cortical representation. Notably, the primary somatosensory cortex in the post central gyms, receives input from the thalamus, and projects to the secondary somatic sensory cortex in the parietal operculum. There are also efferent connections from the sensory cortex to the motor cortex. Notably, the trigeminal nerve is a very large nerve and 28% of the sensory cortex is devoted to it alone.

The present disclosure is based, in part, upon the discovery that peripheral administration in the region of a peripheral nerve, of a combination of CGRP antagonist and a clostridial derivative can treat (including alleviate and/or prevent) a variety of neurological disorders, including disorders mediated via all projections of the trigeminal nucleus, including but not limited to the thalamus, amygdala, hypothalamus, hippocampus, motor, sensory and visual cortex (FIG. 3) The present disclosure is also based upon the discovery that peripheral administration in the region of a peripheral nerve, a cranial nerve, a ganglion or combinations thereof of a combination of a CGRP antagonist and a clostridial derivative can provide significant and long lasting relief from a variety of neurological and neuropsychiatric disorders.

### Definitions

As used herein, the words or terms set forth below have the following definitions:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition. The combination therapy disclosed herein can be locally administered by various methods. For example, intramuscular, intradermal, subcutaneous administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“Biological activity” describes the beneficial or adverse effects of a drug on living matter. When a drug is a complex chemical mixture, this activity is exerted by the substance's active ingredient but can be modified by the other constituents. Biological activity of a clostridial derivative such as a botulinum toxin can be assessed as potency or as toxicity by an in vivo LD₅₀ or ED₅₀ assay, or through an in vitro assay such as, for example, cell-based potency assays as described in U.S. 20100203559 and U.S. 20100233802.

“Botulinum toxin” means a neurotoxin produced by Clostridium botulinum, as well as a botulinum toxin, fragments, variants or chimeras thereof made recombinantly by a non-Clostridial species. The phrase “botulinum toxin”, as used herein, encompasses Botulinum toxin serotype A (BoNT/A), Botulinum toxin serotype B (BoNT/B), Botulinum toxin serotype C (BoNT/C), Botulinum toxin serotype D (BoNT/D), Botulinum toxin serotype E (BoNT/E), Botulinum toxin serotype F (BoNT/F), Botulinum toxin serotype G (BoNT/G), Botulinum toxin serotype H (BoNT/H), Botulinum toxin serotype X (BoNT/X), Botulinum toxin serotype En (BoNT/En), and mosaic Botulinum toxins and/or their subtypes and any other types of subtypes thereof, or any re-engineered proteins, analogs, derivatives, homologs, parts, sub-parts, variants, or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“CGRP”, abbreviated for Calcitonin-Gene-Related-Peptide, as used herein encompasses any member of the calcitonin family, including any calcitonin gene related peptide and analogs, calcitonin, amylin, adrenomedullin and their analogs.

“CGRP antagonist” refers to any molecule that exhibits any one or more of the following characteristics: (a) bind to CGRP or CGRP-R and the binding results in a reduction or inhibition of CGRP activity; (b) block CGRP from binding to its receptor(s); (c) block or decrease CGRP receptor activation; (d) inhibit CGRP biological activity or downstream pathways mediated by CGRP signaling function; (e) increase clearance of CGRP; and (f) inhibit or reduce CGRP synthesis, production or release. CGRP antagonists include but are not limited to antibodies to CGRP, antibodies to the CGRP-R, small molecules that antagonize CGRP, and small molecules that antagonize CGRP-R.

“Clostridial derivative” refers to a molecule which contains any part of a clostridial toxin as defined herein. As used herein, the term “clostridial derivative” encompasses native or recombinant neurotoxins, recombinant modified toxins, fragments, chimeras and variants thereof, a Targeted Vesicular Exocytosis Modulator (TEM), or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/Ci, a BoNT/D, a BoNT/E, a BoNT/F, a BoNT/G, BoNT/H, a BoNT/En, BoNT/X, mosaic botulinum toxins, a Tetanus toxin (TeNT), a Baratii toxin (BaNT), and a Butyricum toxin (BuNT). The BoNT/C2 cytotoxin and BoNT/C3 cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/Ci complex, a 500-kDa BoNT/D complex, a 300-kDa BoNT/D complex, a 300-kDa BoNT/E complex, and a 300-kDa BoNT/F complex.

“Clostridial toxin active ingredient” refers to a molecule which contains any part of a clostridial toxin that exerts an effect upon or after administration to a subject or patient. As used herein, the term “clostridial toxin active ingredient” or “clostridial derivative” encompasses a Clostridial toxin complex comprising the approximately 150-kDa Clostridial toxin and other proteins collectively called non-toxin associated proteins (NAPs), the approximately 150-kDa Clostridial toxin alone, or a modified Clostridial toxin, such as, e.g., a re-targeted Clostridial toxins.

“Combination therapy” refers to a treatment wherein a botulinum toxin and a CGRP antagonist are administered either simultaneously or sequentially, by a similar administration route or by different administration routes.

“Effective amount” as applied to the biologically active ingredient means that amount of the ingredient which is generally sufficient to effect a desired change in the subject. For example, where the desired effect is a reduction in duration or intensity of a neurological or neuropsychiatric disorder and related symptoms, an effective amount of the ingredient is that amount which causes at least a substantial reduction in duration or intensity of the neurological or neuropsychiatric disorder and related symptoms, and without resulting in significant toxicity.

“Intramuscular” or “intramuscularly” means into or within (as in administration or injection of a CGRP antagonist into) a muscle.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Topical administration is a type of local administration in which a pharmaceutical agent is applied to a patient's skin.

“Modified botulinum toxin” means a botulinum toxin that has had at least one of its amino acids deleted, modified, or replaced, as compared to a native botulinum toxin. Additionally, the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter, such as substance P.

“Peripheral administration” means administration by means of a peripheral location on a mammal. Peripheral administration includes subdermal, intranasal, intramuscular, intradermal, transdermal, and subcutaneous administration.

“Pharmaceutical composition” means a composition comprising an active pharmaceutical ingredient, such as, for example, a clostridial toxin active ingredient such as a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. It is also envisioned that a pharmaceutical composition comprising a Clostridial toxin active ingredient can include one or more pharmaceutically acceptable excipients that facilitate processing of an active ingredient into pharmaceutically acceptable compositions. Insofar as any pharmacologically acceptable excipient is not incompatible with the Clostridial toxin active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

“Stabilizing agent”, “stabilization agent” or “stabilizer” means a substance that acts to stabilize a Clostridial toxin active ingredient.

“Stabilizers” can include excipients, and can include protein and non-protein molecules.

“TEM” as used herein, is synonymous with “Targeted Exocytosis Modulator” or “retargeted endopeptidase.” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

“Therapeutic formulation” means a formulation can be used to treat and thereby alleviate a disorder or a disease, such as, for example, a neurological or neuropsychiatric disorder and/or associated symptoms.

“Topical administration” excludes systemic administration of the neurotoxin. In other words, and unlike conventional therapeutic transdermal methods, topical administration of botulinum toxin does not result in significant amounts, such as the majority of, the neurotoxin passing into the circulatory system of the patient.

“Treating” means to alleviate (or to eliminate) a neurological or neuropsychiatric disorder and/or symptoms thereof. “Treating” also encompasses reducing the intensity, duration or frequency of occurrence of the neurological or neuropsychiatric disorder and/or symptoms thereof.

“Variant” means a clostridial neurotoxin, such as wild-type botulinum toxin serotype A, B, C, D, E, F, r G, H, X, En or mosaic botulinum toxins that has been modified by the replacement, modification, addition or deletion of at least one amino acid relative to wild-type botulinum toxin, which is recognized by a target cell, internalized by the target cell, and catalytically cleaves a SNARE (SNAP (Soluble NSF Attachment Protein) Receptor) protein in the target cell.

An example of a variant neurotoxin component can comprise a variant light chain of a botulinum toxin having one or more amino acids substituted, modified, deleted and/or added. This variant light chain may have the same or better ability to prevent exocytosis, for example, the release of neurotransmitter vesicles. Additionally, the biological effect of a variant may be decreased or increased compared to the parent chemical entity. For example, a variant light chain of a botulinum toxin type A having an amino acid sequence removed may have a shorter biological persistence than that of the parent (or native) botulinum toxin type A light chain.

In some embodiments, the clostridial derivative of the present method includes a native, recombinant clostridial toxin, recombinant modified toxin, fragments thereof, targeted exocytosis modulators (TEMs), or combinations thereof. In some embodiments, the clostridial derivative is a botulinum toxin. In alternative embodiments, the clostridial derivative is a TEM.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

In some embodiments, the clostridial derivative is a botulinum toxin, which is selected from the group consisting of botulinum toxin types A, B, Ci, D, E, F, G, H, X, En and mosaic botulinum toxins. In one embodiment, the clostridial derivative of the present method is a botulinum toxin type A. The botulinum toxin can be a recombinant botulinum neurotoxin, such as botulinum toxins produced by E. coli.

The clostridial derivative, such as a botulinum toxin, for use according to the present invention can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. Acceptable excipients or stabilizers include protein excipients, such as albumin or gelatin, or the like, or non-protein excipients, including poloxamers, saccharides, polyethylene glycol, or the like. In embodiments containing albumin, the albumin can be, for example, human serum albumin or recombinant human albumin, or the like. The lyophilized material can be reconstituted with a suitable liquid such as, for example, saline, water, or the like to create a solution or composition containing the botulinum toxin to be administered to the patient.

In some embodiments, to increase the resident time of the clostridial derivative in the joint, the clostridial derivative is provided in a controlled release system comprising a polymeric matrix encapsulating the clostridial derivative, wherein fractional amount of the clostridial derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety. The therapeutically effective amount of the clostridial derivative, for example a botulinum toxin, administered according to the present method can vary according to the potency of the toxin and particular characteristics of the condition being treated, including its severity and other various patient variables including size, weight, age, and responsiveness to therapy. The potency of the toxin is expressed as a multiple of the LD₅₀ value for the mouse, one unit (U) of toxin being defined as being the equivalent amount of toxin that kills 50% of a group of 18 to 20 female Swiss-Webster mice, weighing about 20 grams each.

The therapeutically effective amount of the botulinum toxin according to the present method can vary according to the potency of a particular botulinum toxin, as commercially available Botulinum toxin formulations do not have equivalent potency units. For example, one unit of BOTOX® (onabotulinumtoxinA), a botulinum toxin type A available from Allergan, Inc., has a potency unit that is approximately equal to 3 to 5 units of DYSPORT® (abobotulinumtoxinA), also a botulinum toxin type A available from Ipsen Pharmaceuticals. In some embodiments, the amount of abobotulinumtoxinA, (such as DYSPORT®), administered in the present method is about three to four times the amount of onabotulinumtoxinA (such as BOTOX®) administered, as comparative studies have suggested that one unit of onabotulinumtoxinA has a potency that is approximately equal to three to four units of abobotulinumtoxinA. MYOBLOC®, (known as NEUROBLOC® outside the United States) a botulinum toxin type B available from Elan, currently USWorldmeds, has been reported to have a much lower potency unit relative to BOTOX®. In some embodiments, the botulinum neurotoxin can be a pure toxin, devoid of complexing proteins, such as XEOMIN® (incobotulinumtoxinA). The quantity of toxin administered and the frequency of its administration will be at the discretion of the physician responsible for the treatment and will be commensurate with questions of safety and the effects produced by a particular toxin formulation. In some embodiments, the Clostridial derivative is selected from onabotulinumtoxinA, incobotulinumtoxinA, abotulinumtoxinA, daxibotulinumtoxinA, prabotulinumtoxinA, and rimabotulinumtoxinB.

Without wishing to be bound by theory a physiological mechanism can be set forth to explain the efficacy of the peripheral administration of CGRP antagonist and botulinum toxin. Peripheral administration of a combination of a CGRP antagonist and a botulinum toxin in the region of a peripheral nerve according to the methods disclosed herein is believed to permit the CGRP antagonist and botulinum toxin to either be administered to a site in the region of a patient's cranium, neck or shoulder, and/or to reduce afferent, sensory input from a site in the region of the patient's cranium, neck or shoulder, to thereby influence intracranial neurons involved in a neurological or neuropsychiatric disorder and related symptoms. In addition, the combination allows for lower doses of both and/or each component. This results in decreased side effects. Furthermore, efficacy is increased by having a multimodal mechanism of action from the combination of therapeutic agents. CGRP is only one of many neuro-transmitters released by the peripheral nerves and botulinum toxins have the ability to block more than CGRP release. CGRP antagonists will have enhanced action on neurological diseases, including neurological or neuropsychiatric disorder and related symptoms, by combination with botulinum toxins as these will block other neurotransmitters such as substance P and glutamate.

Administration in the region of a peripheral nerve, a cranial nerve, or combination therof, including but not limited to a trigeminal, occipital, autonomic, spinal or cervical sensory nerve(s) of a CGRP antagonist in combination with a botulinum toxin in accordance with the present disclosure can also block progression of neurological and neuropsychiatric disorders and related symptoms mediated by repeated sensory input to the cortex from a sensory nerve and also from autonomic nervous system components.

Methods and medicaments for treating a neurological or neuropsychiatric disorder, and related symptoms according to the present disclosure can comprise a CGRP antagonist in combination with a clostridial derivative, for example, a botulinum toxin, for peripherally administration in the region of a peripheral nerve of a patient. The CGRP antagonist is administered in a therapeutically effective amount to alleviate at least one symptom of a neurological or neuropsychiatric disorder.

Non-limiting examples of centrally mediated disorders include migraine, epilepsy, chronic pain (such as central sensitization chronic pain, central post stroke pain, regional pain, phantom limb pain, or demyelinating disease pain), reflex sympathetic dystrophy, allodynic states; chronic neurological conditions in which kindling is part of the disease process; mood disorders (including bipolar disease) and movement; muscle-related and neuromuscular disorders.

Neurological Disorders

Epilepsy

Epilepsy describes a condition in which a person has recurrent seizures due to a chronic, underlying process. A seizure is a paroxysmal event due to abnormal, excessive, hypersynchronous discharges from an aggregate of central nervous system neurons. Among the many causes of epilepsy, there are various epilepsy syndromes in which the clinical and pathologic characteristics are distinctive and suggest a specific underlying etiology. The prevalence of epilepsy has been estimated at 5 to 10 people per 1000 population. Severe, penetrating head trauma is associated with up to a 50% risk of leading to epilepsy. Other causes of epilepsy include stroke, infection and genetic susceptibility.

Antiepileptic drug therapy is the mainstay of treatment for most patients with epilepsy and a variety of drugs have been used. See e.g., Fauci, A. S. et al., Harrison's Principles of Internal Medicine, McGraw-Hill, 14.sup.th Edition (1998), page 2321. Twenty percent of patients with epilepsy are resistant to drug therapy despite efforts to find an effective combination of antiepileptic drugs. Surgery can then be an option. Video-EEC monitoring can be used to define the anatomic location of the seizure focus and to correlate the abnormal electrophysiologic activity with behavioral manifestations of the seizure. Routine scalp or scalp-sphenoidal recordings are usually sufficient for localization. A high resolution MRI scan is routinely used to identify structural lesions. Functional Imaging studies such as SPECT and PET are adjunctive tests that can help verify the localization of an apparent epileptogenic region with an anatomic abnormality.

The most common surgical procedure for patients with temporal lobe epilepsy involves resection of the anteromedial temperal lobe (temperal lobotomy) or a more limited removal of the underlying hippocampus and amygdala. Focal seizures arising from extratemporal regions may be suppressed by a focal neocortical resection. Unfortunately, about 5% of patients can still develop clinically significant complications from surgery and about 30% of patients treated with temporal lobectomy will still have seizures.

Chronic Pain

About one third of a population will experience chronic pain. In the United States chronic pain is the most common cause of long-term disability, partially or totally disabling about fifty million people. As the population ages, the number of people needing treatment for chronic pain from back disorders, degenerative joint diseases, rheumatologic conditions, fibromyalgia, visceral diseases, and cancers can be expected to increase.

Various events such as tissue injury can trigger pain signals to the brain. These electrical impulses are carried by thin unmyelinated nerves called nociceptors (C-fibers) that synapse with neurons in the dorsal horn of the spinal cord. From the dorsal horn, the pain signal is transmitted via the spinothalamic tract to the cerebral cortex, where it is perceived, localized and interpreted.

Chronic pain is not just a prolonged version of acute pain. As pain signals are repeatedly generated, neural pathways undergo physiochemical changes that make the central nervous system hypersensitive to the pain signals and resistant to antinociceptive input. This is called central sensitization.

Fibromyalgia is a chronic pain syndrome believed due to central sensitization. Characteristic symptoms of fibromyalgia include widespread pain, fatigue, sleep abnormalities and distress. Patients with fibromyalgia show psychophysical evidence of hyperalgesia, that is a heightened response to mechanical, thermal and electrical stimuli at various tender or trigger points. Treatments for fibromyalgia include steroid trigger point injections and medications such as tricyclic antidepressants, Neurontin, and narcotics, but these all have negative side effects.

Causalgia or Reflex sympathic dystrophy is a chronic pain syndrome following on a traumatic event to the peripheral nerves leading to sensitization with hyperalgesia and allodynia developing in the region of the injured nerve.

Post Stroke Pain

Pain can be debilitating and it is not uncommon to attribute widespread pain in the elderly to osteoarthritis within the spinal column structures and peripheral joints or to other musculoskeletal conditions. However, if pain is widespread and exhibits neuropathic features, such as dysaesthesias (poorly localized burning sensations that occur after a stimulus is applied), allodynia (triggered by stimuli which are not normally painful or pain which occurs other than in the area stimulated), hyperpathia (increased pain from normally painful stimuli) and hyperalgesia, it can be the result of a lesion or disorder such as Thalamic Pain Syndrome or Central Post-Stroke Pain (CPSP) originating from the central nervous system. The source of the pain is via the thalamus, the sensory processing center within the central nervous system.

A stroke is the result of loss of the blood supply to a part of the brain and can result in weakness and slurred speech. CPSP develops in about 8% of stroke patients, occurring within one to six months after the stroke. Common painkillers often have no effect on this pain, although some medications developed for epilepsy and depression may reduce pain after strokes. CPSP has also been treated with intravenous lidocaine or oral opioids, as well as amitriptyline, carbamazepine, and lamotrigine, but these medications have adverse side effects.

Regional Pain Syndrome

Complex Regional Pain Syndrome (CRPS) (also called Reflex Sympathetic Dystrophy Syndrome) is a chronic condition characterized by severe burning pain, pathological changes in bone and skin, excessive sweating, tissue swelling, and extreme sensitivity to touch. The syndrome is a nerve disorder that occurs at the site of an injury (most often to the arms or legs), and the disorder is unique in that it simultaneously affects the nerves, skin, muscles, blood vessels, and bones. It occurs especially after injuries from high-velocity impacts such as those from bullets or shrapnel. However, it may occur without apparent injury. CRPS is believed to be the result of dysfunction in the central or peripheral nervous systems. CRPS I is frequently triggered by tissue injury; the term describes all patients with the above symptoms but with no underlying nerve injury. Patients with CRPS II experience the same symptoms but their cases are clearly associated with a nerve injury. CRPS can strike at any age but is more common between the ages of 40 and 60, although the number of CRPS cases among adolescents and young adults is increasing. CRPS affects both men and women, although most experts agree that it is more common in young women. One visible sign of CRPS near the site of injury is warm, shiny red skin that later becomes cool and bluish.

The pain that patients report is out of proportion to the severity of the injury and gets worse, rather than better, over time. Eventually the joints become stiff from disuse, and the skin, muscles, and bone atrophy. The symptoms of CRPS vary in severity and duration, and early treatment often results in remission. If treatment is delayed, however, the disorder can quickly spread to the entire limb, and changes in bone and muscle may become irreversible. In 50 percent of CRPS cases, pain persists longer than 6 months and sometimes for years. Physicians use a variety of drugs to treat CRPS. Elevation of the extremity and physical therapy are also used to treat CRPS. Injection of a local anesthetic is usually the first step in treatment. TENS (transcutaneous electrical stimulation), a procedure in which brief pulses of electricity are applied to nerve endings under the skin, has helped some patients in relieving chronic pain. In some cases, surgical or chemical sympathectomy (interruption of the affected nerve(s) of the sympathetic nervous system) is performed to relieve pain, but these treatments may also destroy other sensations as well.

Phantom Limb Pain

Phantom limb pain is a conscious feeling of a painful limb, after the limb has been amputated. The brain creates a “whole body map” which remains intact even when a piece of the body no longer exists and phantom sensation or pain can result when the brain sends persistent messages to limbs not there. Phantom pain or sensations can range in type and intensity. For example, a mild form might be experienced as a sharp, intermittent stabbing pain causing the limb to jerk in reaction to the pain. An example of a more severe type might be the feeling that the missing limb is being crushed. Usually phantom limb pain diminishes in frequency and intensity over time. For a small number of amputees, however, phantom limb pain can become chronic and debilitating because of the frequency and severity of the pain. Anesthetics such as lidocaine, marcaine, novocaine, pontocaine, and xylocaine are often used to prevent nerve cells from transmitting pain messages, thus relieving trigger points and reducing stump pain, but their effects are temporary. Anti-inflammatories (acetaminophen, aspirin, ibuprofen), antidepressants (Amitriptyline, Pamelor, Paxil, Prozac, Zoloft), anticonvulsants (Tegratol, Neurontin) and narcotics (Codeine, Demerol, Morphine, Percodan, Percocet) are other medications also used to treat phantom pain, but these often have adverse side effects.

Demyelinating Disease Pain

Demyelinating diseases such as Multiple Sclerosis (MS), progressive multifocal leukoencephalopathy (PML), disseminated necrotizing leukoencephalopathy (DNL), acute disseminated encephalomyelitis, and Schilder disease are acquired chronic, inflammatory diseases that result in the destruction of myelin, the fatty insulation normally covering the nerve fibers that aids in the transmission of nerve impulses. Demyelination results in impaired transmission of action potentials along exposed axons, producing a multiplicity of neurological deficits, for example, sensory loss, weakness, visual loss, vertigo, incoordination, sphincter disturbances, and altered cognition. MS is usually characterized by a relapsing-remitting course in the early stages, with full or nearly full recovery, initially. Over time the disease enters an irreversible progressive phase of neurological deficit. Acute relapses are caused by inflammatory demyelination, while disease progression is thought to result from axonal loss. The disease process affects myelinated fiber tracts, such as the optic nerves and the white matter tracts of the brain and spinal cord. This may lead to a variety of symptoms, such as visual disturbances, bladder, bowel or sexual dysfunction, motor weakness and spasticity, sensory symptoms (numbness, dysaesthesia), cerebellar symptoms (tremor and ataxia), and other symptoms (fatigue, cognitive impairment and psychiatric complications). Therapies used to treat demyelinating disorders can be categorized into disease modifying therapies, drugs used in acute exacerbations and drugs used to treat disease complications. So far, no disease modifying therapy has been found that halts disease progression or improves neurological status.

For this reason, the mainstay of treatment remains symptomatic management. Current therapies predominantly influence the immune system and target the inflammatory processes that are involved in the disease pathology. Beta interferons (interferon beta-1 b, known as Betaferon), glatiramer acetate (Copaxone) and mitozantrone have been used for their immunomodulatory effects. These include inhibition of leukocyte proliferation and antigen presentation, inhibition of T-cell migration across the blood-brain barrier and modulation of cytokine production to produce an anti-inflammatory environment. Oral steroids, such as prednisolone, may be effective in shortening acute attacks of MS. Other potential therapies are undergoing clinical evaluation, including T-cell vaccination, interleukin 10, matrix metalloproteinase inhibitors, plasmapheresis, vitamin D, retinoic acid, ganciclovir, valaciclovir, bone marrow transplantation and autologous stem cell transplantation.

Migraine

Migraine is a disorder where there is dysfunction of brainstem inhibition, which leads to activation of the trigeminal nerve. The branches that supply the meningeal blood vessels release neurotransmitters that include CGRP around these blood vessels. This leads to neurogenic inflammation as the CGRP causes vasodilation and increased vascular permeability. An inflammatory exudate builds up around the blood vessels. This correlates with the migraine headache symptoms of throbbing headache made worse by head movement. The inflammatory causes the trigeminal nerve to fire and this in turn leads to a decreased threshold for nerve activation with resultant sensitization. The trigeminal nerve impulses travel back to the brainstem to the Trigeminal Nucleus Caudalis, and from here second order neurons travel in the trigeminao-thalamic tract to the thalamus. As the thalamus becomes activated the migraine patients develop allodynia, which involves sensitivity to light touch around the head and neck areas. With this symptom light touch is perceived as pain. This usually develops an hout into the headache phase. Triptans are used to vaso-constrict meningeal blood vessels and decrease trigeminal nerve activation. Botulinum toxin injections around extra-cranial trigeminal and occipital nerve endings may also lead to decreased trigeminal nerve activation. CGRP antagonists including monoclonal antibodies to CGRP may decrease the effects of CGRP in the migraine cascade.

As indicated, various therapeutic treatments are available as treatments for various neurological disorders, such as thalamically mediated disorders. However, these therapeutic treatments have several adverse effects. These side-effects may be attributed to the fact that the pharmaceutical agents are typically administered systemically, and therefore, the agents have a relatively non-specific action with respect to the various biological systems of the patient. For example, administration of benzodiazepines may result in sedation and muscle relaxation. In addition, tolerance may develop to these drugs, as well as withdrawal seizures may develop. Current therapeutic strategies also require consistent and repeated administration of the agents to achieve the desired effects.

Neuropsychiatric Disorders

A neuropsychiatric disorder is a neurological disturbance that is typically labeled according to which of the four mental faculties is affected. For example, one group of neuropsychiatric disorders includes disorders of thinking and cognition, such as schizophrenia and delirium. A second group of neuropsychiatric disorders includes disorders of mood, such as depression, affective disorders and anxiety. A third group of neuropsychiatric disorders includes disorders of social behavior, such as character defects and personality disorders. And a fourth group of neuropsychiatric disorders includes disorders of learning, memory, and intelligence, such as mental retardation and dementia. Accordingly, neuropsychiatric disorders encompass schizophrenia, delirium, Alzheimer's disease, depression, mania, attention deficit disorders, drug addiction, dementia, agitation, apathy, anxiety, psychoses, personality disorders, bipolar disorders, obsessive-compulsive disorders, eating disorders, post-traumatic stress disorders, irritability, and disinhibition.

Depression

Major depressive disorder (MDD) (also known as recurrent depressive disorder, clinical depression, major depression, unipolar depression, unipolar disorder, or simply “depression”) is a mental disorder characterized by an all-encompassing low mood accompanied by low self-esteem, and by loss of interest or pleasure in normally enjoyable activities. This cluster of symptoms (syndrome) was named, described and classified as one of the mood disorders in the 1980 edition of the American Psychiatric Association's diagnostic manual. The term “depression” is ambiguous. It is often used to denote this syndrome but may refer to any or all of the mood disorders. Major depressive disorder is a disabling condition which adversely affects a person's family, work or school life, sleeping and eating habits, and general health. In the United States, around 3.4% of people with major depression commit suicide, and up to 60% of people who commit suicide had depression or another mood disorder.

The diagnosis of MDD is based on the patient's self-reported experiences, behavior reported by relatives or friends, and a mental status examination. Currently, there is no laboratory test for major depression, although physicians generally request tests for physical conditions that may cause similar symptoms. If MDD is not detected in the early stages it may result in a slow recovery and affect or worsen the person's physical health. The most common time of onset is between the ages of 20 and 30 years, with a later peak between 30 and 40 years.

Typically, patients are treated with antidepressant medication and, in many cases, also receive psychotherapy or counseling although the effectiveness of medication for mild or moderate cases is questionable. Hospitalization may be necessary in cases with associated self-neglect or a significant risk of harm to self or others. A minority are treated with electroconvulsive therapy (ECT), under a short-acting general anaesthetic. The course of the disorder varies widely, from one episode lasting weeks to a lifelong disorder with recurrent major depressive episodes. Depressed individuals have shorter life expectancies than those without depression, in part because of greater susceptibility to medical illnesses and suicide.

Schizophrenia

Schizophrenia is a disorder that affects about one percent of the world population. Three general symptoms of schizophrenia are often referred to as positive symptoms, negative symptoms, and disorganized symptoms. Positive symptoms can include delusions (abnormal beliefs), hallucinations (abnormal perceptions), and disorganized thinking. The hallucinations of schizophrenia can be auditory, visual, olfactory, or tactile. Disorganized thinking can manifest itself in schizophrenic patients by disjointed speech and the inability to maintain logical thought processes. Negative symptoms can represent the absence of normal behavior. Negative symptoms include emotional flatness or lack of expression and can be characterized by social withdrawal, reduced energy, reduced motivation, and reduced activity. Catatonia can also be associated with negative symptoms of schizophrenia. The symptoms of schizophrenia should continuously persist for a duration of about six months in order for the patient to be diagnosed as schizophrenic. Based on the types of symptoms a patient reveals, schizophrenia can be categorized into subtypes including catatonic schizophrenia, paranoid schizophrenia, and disorganized schizophrenia.

Examples of antipsychotic drugs that may be used to treat schizophrenic patients include phenothizines, such as chlorpromazine and trifluopromazine; thioxanthenes, such as chlorprothixene; fluphenazine; butyropenones, such as haloperidol; loxapine; mesoridazine; molindone; quetiapine; thiothixene; trifluoperazine; perphenazine; thioridazine; risperidone; dibenzodiazepines, such as clozapine; and olanzapine. Although these agents may relieve the symptoms of schizophrenia, their administration can result in undesirable side effects including Parkinson's disease-like symptoms (tremor, muscle rigidity, loss of facial expression); dystonia; restlessness; tardive dyskinesia; weight gain; skin problems; dry mouth; constipation; blurred vision; drowsiness; slurred speech and agranulocytosis.

Mania

Mania is a sustained form of euphoria that affects millions of people in the United States who suffer from depression. Manic episodes can be characterized by an elevated, expansive, or irritable mood lasting several days, and is often accompanied by other symptoms, such as, overactivity, over-talkativeness, social intrusiveness, increased energy, pressure of ideas, grandiosity, distractibility, decreased need for sleep, and recklessness. Manic patients can also experience delusions and hallucinations.

Depressive disorders can involve serotonergic and noradrenergic neuronal systems based on current therapeutic regimes that target serotonin and noradrenalin receptors. Serotonergic pathways originate from the raphe nuclei of the brain stem, and noradrenergic pathways originate from the locus ceruleus. Decreasing the electrical activity of neurons in the locus ceruleus can be associated with the effects mediated by depression medications.

Mania may result from an imbalance in certain chemical messengers within the brain. It has been proposed that mania is attributed to a decline in acetylcholine. A decline in acetylcholine may result in a relatively greater level of norepinephrine. Administering phosphotidyl choline has been reported to alleviate the symptoms of mania.

Anxiety

Anxiety disorders may affect between approximately ten to thirty percent of the population, and can be characterized by frequent occurrence of symptoms of fear including arousal, restlessness, heightened responsiveness, sweating, racing heart, increased blood pressure, dry mouth, a desire to run or escape, and avoidance behavior. Generalized anxiety persists for several months, and is associated with motor tension (trembling, twitching, muscle aches, restlessness); autonomic hyperactivity (shortness of breath, palpitations, increased heart rate, sweating, cold hands), and vigilance and scanning (feeling on edge, exaggerated startle response, difficult in concentrating). benzodiazepines, which enhance the inhibitory effects of the gamma aminobutyric acid (GABA) type A receptor, are frequently used to treat anxiety. Buspirone is another effective anxiety treatment.

Alzheimer's Disease

Alzheimer's disease is a degenerative brain disorder characterized by cognitive and noncognitive neuropsychiatric symptoms, which accounts for approximately 60% of all cases of dementia for patients over 65 years old. Psychiatric symptoms are common in Alzheimer's disease, with psychosis (hallucinations and delusions) present in approximately fifty percent of affected patients. Similar to schizophrenia, positive psychotic symptoms are common in Alzheimer's disease. Delusions typically occur more frequently than hallucinations. Alzheimer's patients may also exhibit negative symptoms, such as disengagement, apathy, diminished emotional responsiveness, loss of volition, and decreased initiative.

Several of the symptoms associated with neuropsychiatric disorders appear to be, at least in part, attributed to hyper-excitability (i.e. sensitization to afferent input from peripheral nerves) of neurons within the brain.

In some embodiments, the disclosure provides for the peripheral administration in the region of a peripheral nerve, of a combination of CGRP antagonists and optionally a clostridial derivative, for example a botulinum toxin, to treat (including alleviate and/or prevent) a variety of neurological or neuropsychiatric disorders and related symptoms. The peripheral administration in the region of a peripheral nerve, a cranial nerve, a ganglion or combinations thereof of a combination of a CGRP antagonist and optionally a botulinum toxin can provide significant and long-lasting relief from a variety of neurological disorders including neurological or neuropsychiatric disorder and related symptoms.

In some embodiments, the clostridial derivative, for example a botulinum toxin, is administered to a trigeminal nerve. Trigeminal nerve administration of botulinum toxins has been disclosed for example in U.S. Pat. Nos. 8,609,112; 8,609,113; 8,734,810; 8,717,572; 9,238,061 and 10,064,921; each of which is hereby incorporated by reference in its entirety.

In some embodiments, the clostridial derivative, for example a botulinum toxin, is administered to a suture line. Suture line administration of botulinum toxins has been disclosed for example in U.S. Pat. Nos. 8,617,571; 9,248,168; 9,827,297; and 10,220,079; each of which is hereby incorporated by reference in its entirety.

### EXAMPLES

The following non-limiting examples provide those of ordinary skill in the art with possible case scenarios and specific methods to treat conditions within the scope of the present disclosure and are not intended to limit the scope of the disclosure. In the following examples administration of a CGRP antagonist in combination with a botulinum toxin can be carried out. For example, by topical application (cream or transdermal patch), subcutaneous injection, or subdermal implantation of a controlled release implant.

### Example 1

A 36 year old female patient presents with rapid cycling bipolar disease, requiring frequent hospitalizations for mania.

She has been unable to tolerate her medications and is poorly compliant with oral medications such as anticonvulsants (Topiramate, Depakote, Lamictal) and Lithium.

She does not want to have Botox injected in her forehead as she works as a stand-up comedian and wants to keep her facial expression.

She is treated with a combination of CGRP monoclonal antibodies and Onabotulinumtoxin A injected along the course of the vagal and accessory nerves along their peripheral course. These injections are done bilaterally along the Sternocleidomastoid and trapezius muscles in the region of the vagal and accessory nerve branches. The vagus nerve descends vertically within the carotid sheath posterolateral to the internal and common carotid arteries and medial to the internal jugular vein at the base of the neck. The arteries are avoided in the injection procedure.

A combination of OnabotulinumtoxinA and CGRP monoclonal antibodies are used to result in a lower effective dose of each agent and thus avoid injecting high doses of OnabotulinumtoxinA into non-spastic muscle groups in the neck, thereby reducing the risk of neck weakness.

The patient tolerates the procedure well. Her mania episodes decrease in frequency and she is able to function outside of the hospital setting. After her second treatment 12 weeks later, she is back to work as a comedian.

### Example 2

A 56-year-old woman with major depression, has been treated with oral anti-depressants with poor results. She undergoes Botox treatment for depression with injections into the procures and corrugator muscles. Unfortunately, she develops a significant brow ptosis and complains that she constantly looks fatigued. She also notes a tightness in her forehead muscles when she attempts to lift her eyebrows which causes her to feel like she has a constant low-level headache. She prefers the option of not taking a medication every day.

She is treated with low dose OnabotulinumtoxinA: only 5 units in each corrugator and 5 units in the procures. In addition, she is also treated with a local injection of CGRP antagonist (monoclonal antibody) into these same muscles with infiltration around the supra-orbital and supra-trochlear nerves which are both branches of the Trigeminal nerve. Following this treatment, her normal facial expression is maintained and her depression lifts. She is re-treated every 12 weeks.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

1. A method for treating, alleviating or reducing the intensity or frequency of occurrence of a neurological disorder or a neuropsychiatric disorder in a patient in need thereof, the method comprising administering a CGRP antagonist in combination with a clostridial derivative to a peripheral nerve, a cranial nerve, or combinations thereof, thereby treating, alleviating or reducing the intensity or frequency of occurrence of the neurological or neuropsychiatric disorder.

2. The method of claim 1, wherein the neurological disorder is selected from the group consisting of migraine, epilepsy, chronic pain, post stroke pain, regional pain syndrome, phantom limb pain, demyelinating disease pain, or combinations thereof.

3. The method of claim 1, wherein the CGRP antagonist is an antibody to CGRP, a small molecule CGRP antagonist; or combinations thereof.

4. The method of claim 1, wherein the CGRP antagonist is an antibody to a CGRP receptor.

5. The method of claim 1, wherein the CGRP antagonist is administered locally.

6. The method of claim 5, wherein the CGRP antagonist is administered by injection.

7. The method of claim 5, wherein the CGRP is administered subcutaneously.

8. The method of claim 1, wherein the clostridial derivative is a botulinum toxin.

9. The method of claim 1, wherein the clostridial derivative is onabotulinumtoxinA.

10. The method of claim 1, wherein the neuropsychiatric disorder is selected from the group consisting of schizophrenia, depression, mania, autism or combinations thereof.

11. A method for treating, alleviating or reducing the intensity or frequency of occurrence of a neurological disorder or a neuropsychiatric disorder in a patient in need thereof, the method comprising administering a CGRP antagonist, and optionally a clostridial derivative to a peripheral nerve, a cranial nerve, or combinations thereof, thereby treating, alleviating or reducing the intensify or frequency of occurrence of the neurological or neuropsychiatric disorder.

12. The method of claim 11, wherein the neurological disorder is selected from the group consisting of migraine, epilepsy, chronic pain, post stroke pain, regional pain syndrome, phantom limb pain, demyelinating disease pain, or combinations thereof.

13. The method of claim 11, wherein the CGRP antagonist is an antibody to CGRP, a small molecule CGRP antagonist; or combinations thereof.

14. The method of claim 11, wherein the CGRP antagonist is an antibody to a CGRP receptor.

15. The method of claim 11, wherein the CGRP antagonist is administered locally.

16. The method of claim 15, wherein the CGRP antagonist is administered by injection.

17. The method of claim 15, wherein the CGRP is administered subcutaneously.

18. The method of claim 11, wherein the clostridial derivative is a botulinum toxin.

19. The method of claim 11, wherein the clostridial derivative is onabotulinumtoxinA.

20. The method of claim 11, wherein the neuropsychiatric disorder is selected from the group consisting of schizophrenia, depression, mania, autism or combinations thereof.
